(19) United States
(12) Patent Application Publication
(10) Pub. No.: US 2018/0280785 A1
(43) Pub. Date: Oct. 4, 2018

(54) DECORATIVE PLATE FOR MOUTH GUARDS

(71) Applicant: Battle-ABC, LLC, Omaha, NE (US)

(72) Inventors: Christopher W. Circo, Bennington, NE (US); Anthony Schrager, Omaha, NE (US)

(21) Appl. No.: 15/477,424

(22) Filed: Apr. 3, 2017

(51) Int. Cl. A63B 71/08

(52) U.S. Cl. CPC A63B 71/08; A61F 5/566; A63B 2071/086; A63B 71/085

(57) ABSTRACT

A decorative plate is provided to removably engage a mouth guard to selectively change its appearance. The decorative plate may include a decorative display on its forward surface to provide the mouth guard with a desired, decorative appearance. In another embodiment, a decorative display member is positioned between a rearward surface of the decorative plate and a forward surface of the mouth guard. The decorative plate may be transparent in such embodiments so that the decorative display member is visible through the forward surface of the decorative plate. Various mounting brackets allow the decorative plate to be removably secured with mouth guards of various designs.

### BACKGROUND

Sports-related concussions have increased over the years. The prior art has produced a wide array of protective equipment, such as helmets, mouth guards, and other headgear in an attempt to reduce the number of sports-related concussions. In particular, mouth guards are believed to help prevent concussions as well as protect the user's teeth from damage. Mouth guards are used by athletes of a wide range of ages in a multitude of sports. Mouth guards have taken on a secondary purpose, beyond physical protection for the user. They also become a way to express individuality, team spirit, and attitude through displays of colors, logos, and images on mouth guard surfaces that are visible at times when the mouth guard is worn by the user. Examples of such displays include team color combinations, mascot logos, or other images.

Decorative mouth guards are permanently marked with various displays. The only way to change the display is to replace the mouth guard. In some situations, an athlete may have four or five different mouth guards with different designs for different moods and games. This is impractical and increases costs unreasonably. It leaves users with a single type of mouth guard display to show their team spirit or individuality. Accordingly, there is a need for a simple and cost effective system and method for providing selectively interchangeable mouth guard displays.

### SUMMARY

This Summary is provided to introduce a selection of concepts in a simplified form that are further described below in the Detailed Description. Neither this Summary, nor the foregoing Background, is intended to identify key aspects or essential aspects of the claimed subject matter. Moreover, this Summary is not intended for use as an aid in determining the scope of the claimed subject matter.

Embodiments of decorative plates are presented that may be coupled with mouth guards of various designs to provide users with a manner of expressing team spirit and individuality. In various embodiments, the decorative plates are coupled with a mouth guard having an exterior lip protector. Such mouth guards include a dental tray having opposing biting surfaces that receive the upper and lower arcades of the user's teeth. An inner wall extends outwardly from a peripheral edge of the dental tray. A conduit extends between the inner wall and the outer wall and includes at least one open passage that extends through opposite end portions of the conduit.

Embodiments of the decorative plate include a decorative display on a forward surface. Such a decorative display will include at least one of a color, combination of colors, lettering, numbering, or an image. The decorative plate is operatively, removably coupled with the mouth guard such that the rearward surface of the decorative plate at least partially obscures the forward surface of an outer wall when the decorative plate is coupled with the mouth guard. The forward surface of the outer wall may have no decorative display or it may have a decorative display that the user wishes to change.

Where the mouth guard includes an outer wall opening, embodiments of the decorative plate include at least one decorative plate opening that is in open fluid communication with the outer wall opening and the conduit passage when the decorative plate is coupled with the outer wall. The decorative plate includes means for removably securing the decorative plate with the mouth guard. In some embodiments, the decorative plate includes a mounting bracket that extends outwardly from the rearward face of the decorative plate and is shaped to at least partially reside within the conduit passage. In some embodiments, the mounting bracket is pressure fit within the conduit passage. In other embodiments, the mounting bracket is formed from a plurality of laterally spaced-apart fingers that extend from the rearward opening of the conduit passage when the decorative plate and mouth guard are coupled with one another. Bracket teeth extend outwardly from distal end portions of the fingers and are positioned to engage portions of the mouth guard outside of the conduit passage. The decorative plate can be secured to the outer wall by other types of mounting brackets. In still another embodiment, an adhesive may be disposed on one or both of the decorative plate or the outer wall.

In some embodiments, a decorative display member is positioned between the decorative plate and the outer wall. The decorative display member includes at least one of a color, combination of colors, lettering, numbering, or an image. Where a decorative display member is used, the decorative plate is be formed from a material that is sufficiently transparent to allow the decorative display member to be viewed through the forward surface of the decorative plate.

The decorative plate and the present technology are also adaptable for use with mouth guards of other designs. For example, conventional mouth guards that do not have an outer wall that is positioned outside the user's mouth will have an exterior facing wall that is a vertical face of the inner wall from the top of the mouth guard to the bottom. Mouth guards of such design may have a solid surface or may have one or more breathing apertures similar to the conduit passage that passes through the outer face of the inner wall, through to the rearward opening. In such embodiments, the decorative plate can include a mounting bracket, which may be of a one-piece design or having a plurality of fingers. Other methods of securing the decorative plate to the exterior facing wall of the mouth guard are also provided.

These and other aspects of the present system and method will be apparent after consideration of the Detailed Description and Figures herein.

### DRAWINGS

Non-limiting and non-exhaustive embodiments of the mouth guard, including the preferred embodiment, are described with reference to the following figures, wherein like reference numerals refer to like parts throughout the various views unless otherwise specified.

FIG. 1 is a front perspective view of one embodiment of a decorative plate, according to the present technology, and mouth guard to which it may be selectively coupled, in a decoupled position, according to an exemplary embodiment of the present technology;

FIG. 2 is a rear perspective view of the decorative plate shown in FIG. 1;

FIG. 3 is a top plan view of the mouth guard and decorative plate shown in FIG. 1;

FIG. 4A is a partial, cut-away, side elevation view of one embodiment of the decorative plate of the present technology, depicting one manner in which the decorative plate may be aligned with a mouth guard to which it may be secured;

FIG. 4B is a partial, cut-away, side elevation view of the decorative plate and mouth guard of FIG. 4A, depicting one manner in which the decorative plate may be secured with a mouth guard;

FIG. 5A is a partial, cut-away, side elevation view of another embodiment of the decorative plate of the present technology, depicting another manner in which the decorative plate may be aligned with a mouth guard to which it may be secured;

FIG. 5B is a partial, cut-away, side elevation view of the decorative plate and mouth guard of FIG. 5A, depicting another manner in which the decorative plate may be secured with a mouth guard; and

FIG. 6 is a front perspective view of another embodiment of a decorative plate and a decorative display sheet, according to the present technology, and mouth guard to which they may be selectively coupled, in a decoupled position, according to an exemplary embodiment of the present technology.

### DETAILED DESCRIPTION

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense.

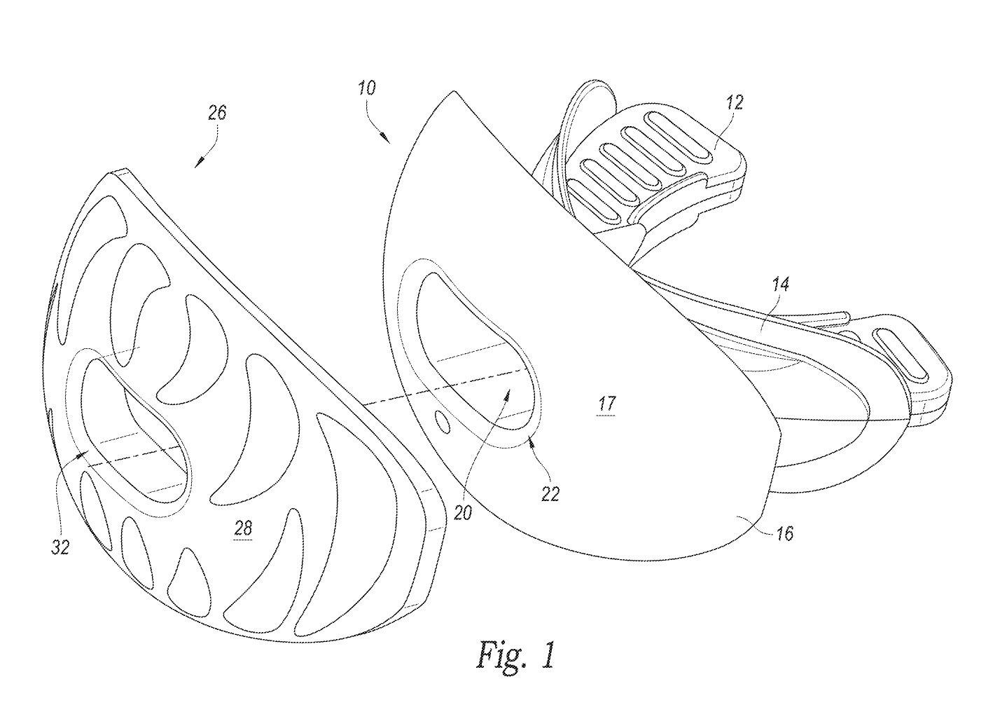

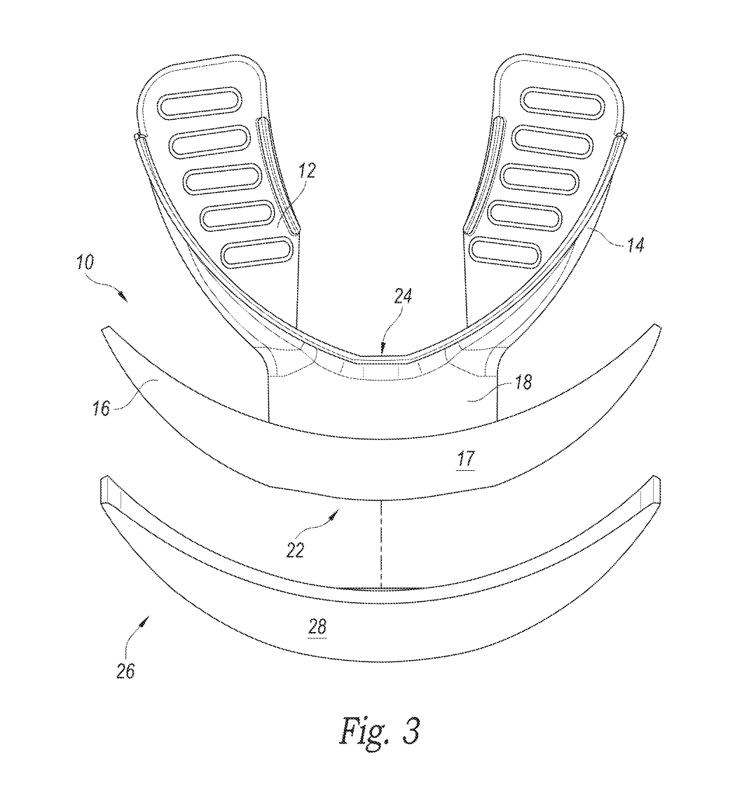

With reference to FIGS. 1-6, decorative plates may be coupled with mouth guards of various designs to provide users with a manner of expressing team spirit and individuality. In various embodiments, the decorative plates of the present technology are coupled with an increasingly popular style of mouth guard having an exterior lip protector. An exemplary embodiment of such a mouth guard is depicted in FIGS. 1, 3, and 6. Such mouth guards 10 include a dental tray 12 that is shaped to have biting surfaces on opposite sides of the dental tray 12 that receive the upper and lower arcades of the user's teeth. It is contemplated that the dental tray 12 may be a continuous U-shape or provided in the form of spaced-apart molar receiving members, such as depicted in the Figures. An inner wall 14 extends outwardly from a peripheral edge of the dental tray and is configured for insertion between a user's lips and teeth. A forward surface of the inner wall 14 is shaped to confront an inner surface of the user's mouth. An opposite, rearward surface of the inner wall 14 is shaped to confront forward surfaces of the user's teeth. An outer wall 16 includes a forward surface 17 and an opposite rearward surface, wherein the rearward surface is shaped to confront an exterior surface of a user's lips. A conduit 18 extends between the inner wall 14 and the outer wall 16 such that the forward surface of the inner wall is longitudinally separated from the rearward surface of the outer wall by a gap defined by a length of the conduit. At least one open passage 20 extends through opposite end portions of the conduit 18. In some embodiments, the passage 20 is a single opening that extends through the conduit 18. In other embodiments, the passage 20 may be partitioned by one or more walls to define a plurality of passages that are at least partially separated from one another. The outer wall 16 includes at least one outer wall opening 22 that penetrates the outer wall 16 and is in open fluid communication with the passage 20. A rearward opening 24 defines an end of the passage 20 that penetrates either or both of the dental tray 12 or the inner wall 14. In some aspects of the mouth guard 10, the passage 20 provides a means whereby a user may breathe or drink through passage 20 while the mouth guard 10 is worn within the user's mouth.

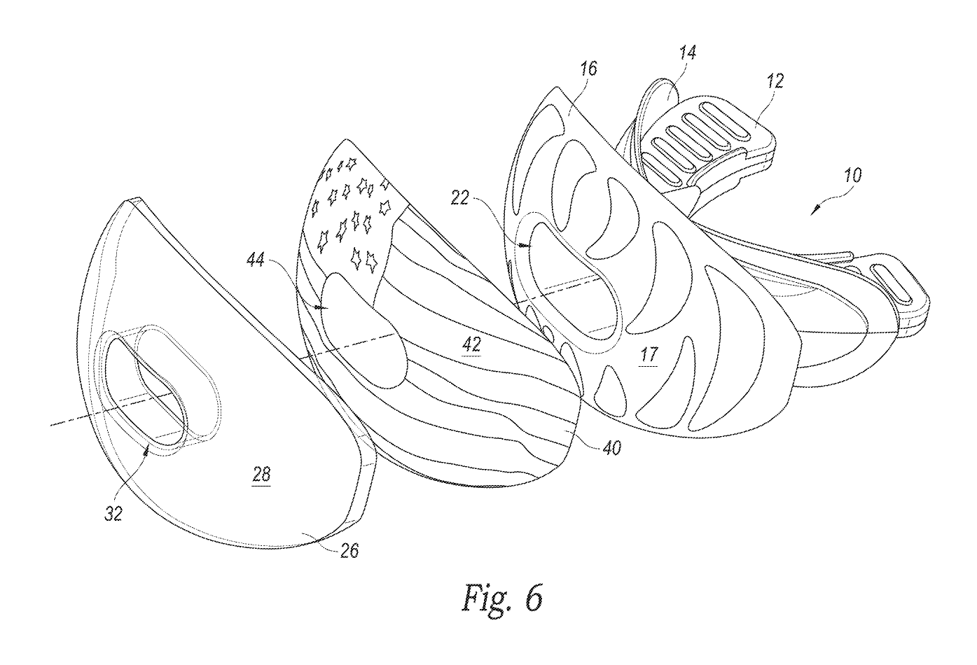

In various embodiments, a decorative plate 26 includes a forward surface 28 and an opposite rearward surface 30. Particular embodiments of the decorative plate 26 include a decorative display on the forward surface 28. It is contemplated that the decorative display on the decorative plate 26 will be limited only by the imagination, with an infinite number of different types of visual adornment. Generally, the decorative display will include at least one of a color, combination of colors, lettering, numbering, or an image. Solid colors and various color combinations, such as team colors provide one manner of decoration. One or more words or slogans, such as team names, or an infinite number of various alpha-numeric combinations provide another manner of decoration. Images, such as drawings, photos, and other depictions, such as the fangs of FIG. 1 or the American Flag of FIG. 6, provide still another manner of decoration. It is contemplated that the aforementioned decorations may be combined with one another to define the decorative display.

The decorative plate 26 is operatively, removably coupled with the mouth guard 10 such that the rearward surface 30 of the decorative plate at least partially obscures the forward surface 17 of the outer wall 16 when the decorative plate 26 is coupled with the mouth guard 10. Accordingly, it is contemplated that the forward surface 17 of the outer wall 16 may have no decorative display or it may have a decorative display, such as described with regard to the forward surface 28 of the decorative plate 26, i.e. at least one of a color, combination of colors, lettering, numbering, or an image. In various embodiments, the forward surface 17 of the outer wall 16 includes a decorative display that is different from the decorative display on the forward surface 28 of the decorative plate 26. In this manner, the decorative plate 26 selectively changes the appearance of the mouth guard 10 from one visible display to another. The removable nature of the decorative plate 26 permits the user to change the appearance of the mouth guard 10 at will. A plurality of different decorative plates 26 provide the user with a number of different designs from which to choose.

In embodiments where the mouth guard 10 includes an outer wall opening 22, it is contemplated that the decorative plate 26 will include at least one decorative plate opening 32 that penetrates the decorative plate 26 and is in open fluid communication with the outer wall opening 22 and the conduit passage 20 when the decorative plate 26 is coupled with the outer wall 16. It is contemplated that embodiments of the present technology will provide the outer wall opening 22 and the decorative plate opening 32 in similar shapes that align with one another for a clean appearance and full concealment of the outer wall 16 by the decorative plate 26. Although depicted as obround in the figures, the outer wall opening 22 and decorative plate opening 32 could be, for example and without limitation, oval, round, or rectangular, to name a few shapes. In the depicted example, the obround shape is positioned so that a long axis of the shape extends generally parallel to the upper and lower arcades of teeth and the short axis extends perpendicular to the upper and lower arcades of teeth. Accordingly, the size of the passageway is maximized to allow airflow for athletic exertion while limiting the distance that the user's jaw must remain open to accommodate the conduit passage 20.

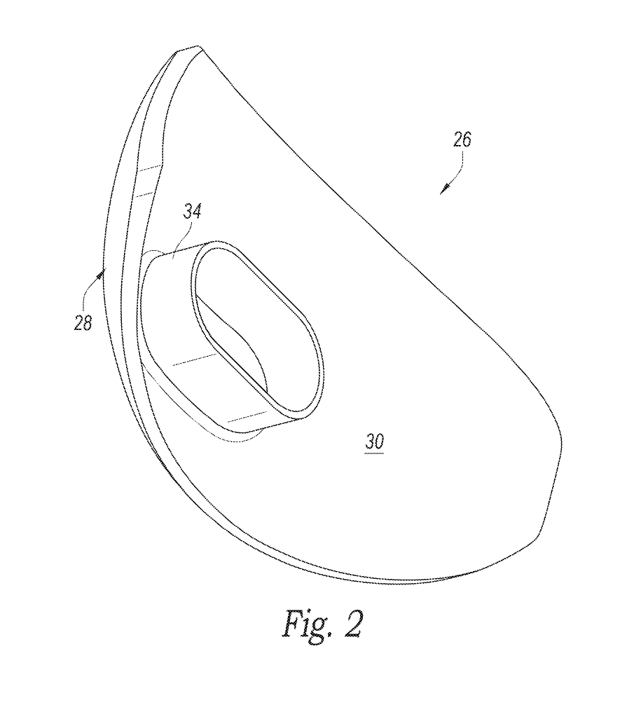

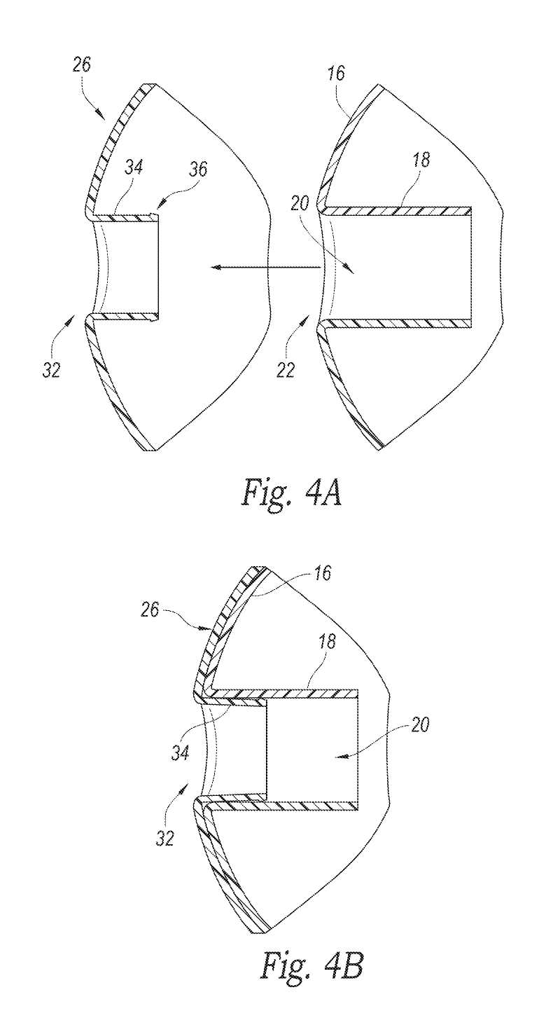

The decorative plate 26 includes means for removably securing the decorative plate 26 with the mouth guard 10. With reference to FIGS. 2, 4A, and 4B, the decorative plate 26 includes a mounting bracket 34 that extends from a peripheral edge of the decorative plate opening 32, outwardly from the rearward face 30 of the decorative plate 26. The mounting bracket 34 is shaped to at least partially reside within the conduit passage 20 when the decorative plate 26 is coupled with the outer wall 16. FIG. 4B depicts an exemplary embodiment where the mounting bracket 34 is pressure fit within the conduit passage 20 when the decorative plate 26 is coupled with the outer wall 16. In such embodiments, the mounting bracket 34 is sized to just fit within the conduit passage 20. In some embodiments, at least one bracket tooth 36 extends outwardly from a distal end portion of the mounting bracket 34. The bracket tooth provides an additional element to resist the unintentional withdrawal of the mounting bracket 34 from within the conduit passage 20. While the mounting bracket 34 is depicted as being a continuous shape with a closed periphery, it is contemplated that the mounting bracket 34 could be formed from a plurality of laterally spaced-apart fingers.

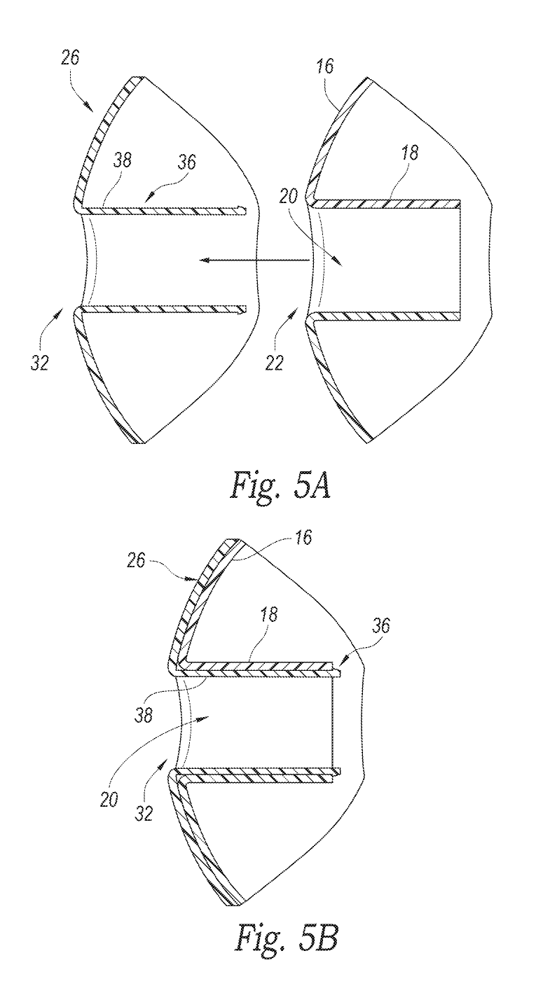

With reference to FIGS. 5A and 5B, an alternate mounting bracket is formed from a plurality of laterally spaced-apart fingers 38 that extend at least partially out from the rearward opening 24 of the conduit passage 20. Bracket teeth 36 extend outwardly from distal end portions of the fingers 38 and are positioned to engage portions of the mouth guard 10 outside of the conduit passage 20, such as the exemplary embodiment depicted in FIG. 5B. In some embodiments, the fingers 38 will have a degree of resilient deformability that allow the fingers 38 to compress inwardly toward one another to allow passage of the fingers through the conduit passage 20 for insertion or removal. While the fingers 38 are depicted as a pair of fingers 38, with one positioned at a top portion of the mounting bracket and another positioned at a bottom portion of the mounting bracket, other configurations are contemplated. For example, the mounting bracket may include a plurality of fingers 38 at the top portion or the mounting bracket and/or the bottom portion of the mounting bracket. Similarly, the fingers 38 may be paired to extend along the opposite side portions of the mounting bracket, in addition to or in place of the fingers 38 at the top and bottom portions of the mounting bracket. When placed at the side portions of the mounting bracket, one or more fingers 38 may be positioned at each side portion of the mounting bracket.

In other embodiments, it is contemplated that the decorative plate 26 can be secured to the outer wall 16 by other means. In one such embodiment, mounting brackets may be provided at the peripheral edges of either or both of the outer wall 16 and decorative plate 26. Such mounting brackets may take any number of forms that simply clasp the opposing structure to mechanically hold the two in a coupled positioned with respect to one another, such as along a reverse or opposite side of the opposing structure. In still another embodiment, an adhesive may be disposed on one or both of the rearward surface 28 of the decorative plate or the forward surface 17 of the outer wall 16. In some embodiments, the adhesive is a pressure sensitive adhesive that can enable the decorative plate 26 and the outer wall 16 to be coupled with one another and decoupled with one another, selectively, multiple times.

With reference to FIG. 6, a decorative display member 40 is positioned between the rearward surface 30 of the decorative plate 26 and the forward surface 17 of the outer wall 16. It is contemplated that the decorative display member 40 could be formed from one of various paper or cardboard materials, thin plastics or metals. Regardless of its construction, the forward surface 42 of the decorative display member 40 includes at least one of a color, combination of colors, lettering, numbering, or an image, similar to that described above with regard to the forward surface 28 of the decorative plate 26. Where a decorative display member 40 is used, the decorative plate 26 will be formed from a material that is sufficiently transparent to allow the decorative display member 40 to be viewed through the forward surface 28 of the decorative plate 26. In such embodiments, it is contemplated that the forward surface of the outer wall 16 will have a decorative display that is different from the decorative display member 40 positioned between the rearward surface 30 of the decorative plate 26 and the forward surface of the outer plate 16. In some embodiments, the decorative display member 40 is removably disposed between the rearward surface 30 of the decorative plate 26 and the forward surface of the outer plate 16. In such instances, the mounting bracket 34 will pass through an opening 44 that penetrates the decorative display member 40 to help align and support the decorative display member 40. In particular embodiments, the decorative display member 40 is adhered to at least one of the rearward surface 30 of the decorative plate 26 or the forward surface 17 of the outer plate 16 with an adhesive.

The decorative plate 26 and the present technology are also adaptable for use with mouth guards of other designs. For example, conventional mouth guards that do not have an outer wall 16 that is positioned outside the user's mouth will have an exterior facing wall that is a vertical face of the inner wall 14 from the top of the mouth guard to the bottom, for example, in the mouth guard depicted in FIG. 1. The decorative plate 26 can be provided with a peripheral edge that is shaped to mimic a peripheral edge of the exterior facing wall. It may also be of a smaller shape to expose portions of the exterior facing wall. Mouth guards of such design may have a solid surface or may have one or more breathing apertures similar to the conduit passage 20 that passes through the outer face of the inner wall, through to the rearward opening 24. In such embodiments, the decorative plate 26 can include a mounting bracket, such as the mounting bracket 34 described herein, which may be of a one-piece design or having a plurality of fingers 38. Other methods of securing the decorative plate to the exterior facing wall of the mouth guard, such as described hereinabove, may be implemented.

The decorative plates, mouth guards, and decorative display members described herein may be formed of a suitable rubber or plastic materials as are known in the art. For example and without limitation, they may be formed from thermoplastic elastomer (TPE) or ethylene vinyl acetate (EVA) or a combination thereof. They may be provided in various degrees of rigidity and flexibility. They may also be formed by any suitable manufacturing process, such as for example, injection molding, insert molding, welding, gluing, and the like. In addition, the decorative plates, mouth guards, and decorative display members described herein may be decorated within mold labeling and in mold decorating techniques, as are known in the art.

Although the structures, technology, and methods of using and/or applying the same have been described in language that is specific to certain structures, materials, and methodological steps, it is to be understood that the present mouth guard is not necessarily limited to the specific structures, materials, and/or steps described. Rather, the specific aspects and steps are described as forms of implementing the disclosed mouth guard. Many embodiments can be practiced without departing from the spirit and scope of the mouth guard described herein. Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques. Moreover, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

What is claimed is:

1. A mouth guard display system comprising:
a mouth guard including:
a dental tray having opposite upper and lower biting surfaces and an inner wall operatively coupled to the molar receiving members, having opposing forward and rearward surfaces; the forward surface of the inner wall being shaped to confront an inner surface of the user's mouth; the rearward surface of the inner wall being shaped to confront forward surfaces of the user's teeth;
an outer wall having opposing forward and rearward surfaces; the rearward surface of the outer wall being shaped to confront an exterior surface of a user's lips;
a conduit extending between the inner wall and outer wall such that the forward surface of the inner wall is longitudinally separated from the rearward surface of the outer wall by a gap defined by a length of the conduit; the conduit having at least one open passage that extends through opposite end portions of the conduit; and
a decorative plate having opposing forward and rearward surfaces; the decorative plate being operatively, removably coupled with the mouth guard such that the rearward surface of the decorative plate at least partially obscures the forward surface of the outer wall when the decorative plate is coupled with the mouth guard.

2. The mouth guard display system of claim 1 wherein the forward surface of the decorative plate includes a decorative display.

3. The mouth guard display system of claim 2 wherein the decorative display is at least one of a color, combination of colors, lettering, numbering, or an image.

4. The mouth guard display system of claim 3 wherein the forward surface of the outer wall includes a decorative display that is different from the decorative display of the decorative plate.

5. The mouth guard display system of claim 2 wherein the decorative display is at least one of a color, combination of colors, lettering, numbering, or an image.

6. The mouth guard display system of claim 1 wherein the forward surface of the outer wall includes a decorative display and the decorative plate is sufficiently transparent to allow the decorative display to be viewed through the forward surface of the decorative plate.

7. The mouth guard display system of claim 1 further comprising:
a decorative display member positioned between the rearward surface of the decorative plate and the forward surface of the outer plate; the decorative plate being sufficiently transparent to allow the decorative display member to be viewed through the forward surface of the decorative plate.

8. The mouth guard display system of claim 7 wherein the decorative display member includes at least one of a color, combination of colors, lettering, numbering, or an image.

9. The mouth guard display system of claim 8 wherein the forward surface of the outer wall includes a decorative display that is different from the decorative display member positioned between the rearward surface of the decorative plate and the forward surface of the outer plate.

10. The mouth guard display system of claim 8 wherein the decorative display member positioned between the rearward surface of the decorative plate and the forward surface of the outer plate is removably disposed between the rearward surface of the decorative plate and the forward surface of the outer plate.

11. The mouth guard display system of claim 8 wherein the decorative display member positioned between the rearward surface of the decorative plate and the forward surface of the outer plate is adhered to at least one of the rearward surfaces of the decorative plate or the forward surface of the outer plate.

12. The mouth guard display system of claim 1 wherein:
the conduit includes at least one conduit passage that extends through opposite end portions of the conduit;
the outer wall having at least one outer wall opening that penetrates the outer wall and is in open fluid communication with the at least one conduit passage;
the decorative plate having at least one decorative plate opening that penetrates the decorative plate and is in open fluid communication with the at least one outer wall opening and the at least one conduit passage when the decorative plate is coupled with the outer wall; and
means for removably securing the decorative plate with the mouth guard.

13. The mouth guard display system of claim 12 wherein the means for removably securing the decorative plate with the mouth guard includes a mounting bracket that extends from a peripheral edge of the at least one decorative plate opening, outwardly from the rearward face of the decorative plate; the mounting bracket being shaped to at least partially reside within the at least one conduit passage when the decorative plate is coupled with the outer wall.

14. The mouth guard display system of claim 13 wherein the mounting bracket is pressure fit within the at least one conduit passage when the decorative plate is coupled with the outer wall.

15. The mouth guard display system of claim 13 wherein the mounting bracket includes a plurality of fingers that extend at least partially out from a rearward opening of the conduit passage; bracket teeth extending outwardly from a distal end portion of the plurality of fingers and positioned to engage a portion of the mouth guard outside of the conduit passage.

16. A method of decoratively adorning a mouth guard, the method comprising:
operatively coupling a decorative plate, having opposing forward and rearward surfaces, with an outer wall of a mouth guard, having opposing forward and rearward surfaces, so that the rearward surface of the decorative plate at least partially obscures the forward surface of the outer wall;
at least one of the forward surface of the decorative plate or the forward surface of the outer wall including a decorative display.

17. The method of claim 16 wherein the decorative display is at least one of a color, combination of colors, lettering, numbering, or an image.

18. The method of claim 16 wherein the forward surface of the decorative plate includes a decorative plate decorative display and the forward surface of the outer wall includes an outer wall decorative display; the decorative plate decorative display being different from the outer wall decorative display.

19. The method of claim 16 further comprising:
positioning a decorative display sheet between the rearward surface of the decorative plate and the forward surface of the outer plate; the decorative plate being sufficiently transparent to allow the decorative display sheet to be viewed through the forward surface of the decorative plate; the decorative display sheet including at least one of a color, combination of colors, lettering, numbering, or an image.

20. A mouth guard display system comprising:
a mouth guard including:
a dental tray having opposite upper and lower biting surfaces and an exterior wall operatively coupled to the molar receiving members, having opposing forward and rearward surfaces; the forward surface of the exterior wall being shaped to confront an inner surface of the user's mouth; the rearward surface of the inner wall being shaped to confront forward surfaces of the user's teeth; and
a decorative plate having opposing forward and rearward surfaces; the decorative plate being operatively, removably coupled with the mouth guard such that the rearward surface of the decorative plate at least partially obscures the forward surface of the exterior wall when the decorative plate is coupled with the mouth guard.
